(19) United States
(12) Patent Application Publication
(10) Pub. No.: US 2016/0030452 A1
(43) Pub. Date: Feb. 4, 2016

(54) Minocycline Derivatives

(71) Applicant: REVANCE THERAPEUTICS, INC., Newark, CA (US)

(72) Inventors: JACOB M. WAUGH, SAN FRANCISCO, CA (US); JACK PHILLIP KENNEDY, NEWARK, CA (US)

(21) Appl. No.: 14/777,110

(22) PCT Filed: Mar. 13, 2014
(86) PCT No.: PCT/US2014/025523

§ 371 Date: Sep. 15, 2015

Related U.S. Application Data
(60) Provisional application No. 61/799,933, filed on Mar. 15, 2013.

(51) Int. Cl. A61K 31/65; C07C 303/28; C07C 231/12; C07C 309/66; A61K 9/00; C07C 237/26

(52) U.S. Cl. CPC A61K 31/65; A61K 9/0053; A61K 9/0014; A61K 9/0019; C07C 237/26; C07C 231/12; C07C 309/66; C07C 303/28

(57) ABSTRACT

This invention relates generally to minocycline derivatives, and to compositions, including pharmaceutical compositions, containing such minocycline derivatives. The invention also relates to methods of synthesizing minocycline derivatives and to methods for using such minocycline derivatives as anti-bacterial agents for treating or preventing infections.

This application claims benefit of priority to U.S. Provisional Patent Application No. 61/799,933, filed on Mar. 15, 2013, the contents of which are hereby incorporated by reference in their entirety.

### FIELD OF THE INVENTION

This invention relates generally to minocycline derivatives, and to compositions, including pharmaceutical compositions, containing such minocycline derivatives. The invention also relates to methods of synthesizing minocycline derivatives and to methods for using such minocycline derivatives as anti-bacterial agents for treating or preventing infections.

### BACKGROUND OF THE INVENTION

Infectious diseases are caused by the presence and growth of pathogens, including, among others, viruses, bacteria, and fungi. Generally, antibiotics are compounds that kill or retard bacteria growth, and are frequently used to treat various forms of infections. Broad-spectrum antibiotics are effective drugs against both Gram-positive and Gram-negative bacteria. Examples of such broad-spectrum antibiotics are ampicillin, amoxicillin, streptomycin, and tetracycline, among others.

Minocycline is a broad spectrum tetracycline-class antibiotic, and the most lipid-soluble member of the class, with a longer half-life than other tetracyclines. Minocycline is one of the most frequently prescribed antibiotics and is often administered in the form of a free base, an acid salt (e.g., hydrochloride salt) or a mixture thereof. Minocycline's structure is shown below.

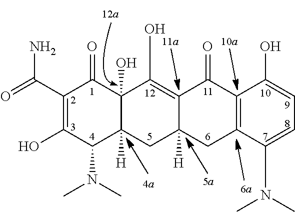 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

Due to its enhanced ability to cross the blood-brain barrier, minocycline is used for the treatment of numerous inflammatory and non-inflammatory infectious diseases, such as acne, methicillin-resistant Staphylococcus aureus (MRSA), and Lyme disease. Minocycline's antibacterial and anti-inflammatory properties make it useful for treating, among other things, asthma, rheumatoid arthritis, amoebic dysentery, anthrax, cholera, gonorrhea, Gougerot-Carteaud Syndrome (Confluent and Reticulated Papillomatosis), bubonic plague, perioral dermatitis, periodontal disease, respiratory infections such as pneumonia, HIV (as an adjuvant), Rocky Mountain spotted fever, rosacea, syphilis, urinary tract infections, rectal infections, and skin infections such as Hidradenitis Suppurativa.

While minocycline is useful for treating a wide variety of infections, minocycline is also known to cause certain unwanted side effects in some patients. For instance, minocycline has been reported to cause diarrhea, drowsiness, mouth sores, vomiting, and/or headaches in certain individuals. In addition, minocycline has been reported to cause certain vestibular disturbances, such as vertigo, ataxia, dizziness, and tinnitus. Many of these unwanted side effects have been correlated with high systemic concentrations of minocycline that occur as the result of the initial rapid absorption of minocycline. Accordingly, previous attempts to reduce these side effects have focused on combining minocycline with various excipients to produce formulations that release minocycline more slowly, thereby avoiding side effects associated with high systemic concentrations of minocycline. However, using such excipients invariably add complexity to the manufacturing process. It would be useful, therefore, to have a minocycline formulation that does not require such excipients to minimize the undesirable side effects associated with high systemic concentrations of minocycline.

### SUMMARY OF INVENTION

In one aspect, the present invention provides derivatives of minocycline and compositions, including pharmaceutical compositions, comprising such derivatives. As described herein, the derivatives of minocycline, and metabolites thereof, may be used as antibacterial compounds. In certain embodiments, the derivatives of minocycline act as a prodrug that generates minocycline in the body through normal metabolic pathways, but at a rate that reduces the peak plasma concentration as compared to conventional administration of an equivalent amount minocycline. Without wishing to be bound by theory, it is believed that these properties allow the minocycline derivatives contemplated by the invention to be administered with fewer of the undesirable side effects associated with high systemic minocycline concentrations. The invention also provides methods to treat and/or prevent infections using such derivatives. The invention further provides methods of synthesizing minocycline derivatives.

In general, the minocycline derivatives of the invention are substituted at the hydroxyl of the C₁₀ position. One aspect of the invention provides compounds according to formula (I)

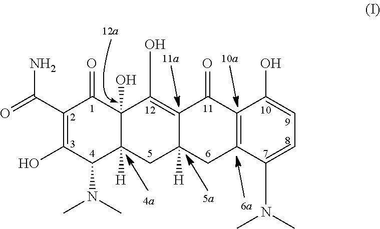

wherein R₁ is selected from an alkyl, substituted alkyl, and heteroaryl. Pharmaceutically acceptable salts tautomers, or stereoisomers of the compounds of formula (I) are also contemplated by the invention.

Another aspect of the invention is to provide pharmaceutical compositions comprising compounds according to formula (I) (or salts, tautomers, or stereoisomers thereof), along with a pharmaceutically acceptable diluent or carrier.

Yet another aspect of the invention is to provide method for synthesizing compounds according to formula (I). The method comprises reacting minocycline with a proton extraction agent and a derivatizing agent, as described herein, and isolating the resulting minocycline derivative.

The invention also provides a method of treating conditions or diseases associated with infections. The method includes administering to an affected individual in need thereof a therapeutically effective amount of a compound according to formula (I), or a pharmaceutically acceptable salt, tautomer, or stereoisomer thereof.

The invention encompasses many possible administration and dosage regimes, with administration strategies including, but not limited to, administration of the minocycline derivatives after the infection is detected or as preventive therapy. Administration schedules may also be altered to achieve a therapeutically effective concentration of the minocyline derivatives to treat the disorder or symptoms described herein.

### DETAILED DESCRIPTION OF THE INVENTION

In one aspect, the invention provides compounds according to formula (I), wherein R₁ is selected from the group consisting of alkyl, substituted alkyl, and heteroaryl.

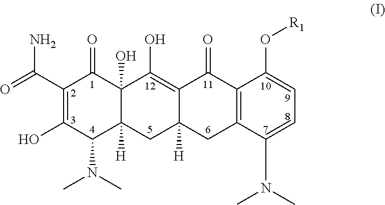

The invention also encompasses pharmaceutically acceptable salts, tautomers, or stereoisomers of the compounds according to formula (I). Polymorphs of the compound are also within the scope of the invention.

As used herein, the term “alkyl” refers to saturated straight, branched chain, or cyclic hydrocarbon radicals derived by the removal of one hydrogen atom from a single carbon atom of the parent alkane. In certain preferred implementations of the invention, the alkyl group contains from 1 to 20 carbon atoms, 1 to 10 carbon atoms, 1 to 5 carbon atoms, or 1 to 3 carbon atoms. Non-limiting examples of typical “alkyl” contemplated by the invention include methyl, ethyl, propan-1-yl, propan-2-yl (isopropyl) cyclopropan-1-yl; butan-1-yl, butan-2-yl (sec-butyl), 2-methyl-propan-1-yl (isobutyl), 2-methyl-propan-2-yl (t-butyl), cyclobutan-1-yl, and the like. In certain preferred implementations of the invention, the alkyl is selected from the group consisting of methyl, ethyl, propan-1-yl, and propan-2-yl. In certain preferred implementations, the alkyl is methyl or ethyl, but preferably methyl.

As used herein, the term “substituted alkyl” refers to an alkyl in which one or more hydrogen atoms are independently replaced with the same or different substituents. Non-limiting examples of substituents within the scope of the invention include —X, —CX₃, —OR², —C(O)R², —C(S)R², —C(O)OR², —C(O)NR²R³, —SR², —S—, ═S, —O—, ═O, —CN, —OCN, —SCN, —NO, NO₂, S(O)₂O, —NR²R³, ═NR², —S(O)₂OH, —S(O)₂R², —R²S(O)₂R³, —OS(O)₂R², —S(O)₂CX₃, wherein each X is independently a halogen, and each R² and R³ are independently hydrogen, alkyl, aryl, substituted aryl, arylalkyl, substituted arylalkyl, and cycloalkyl. In certain cases, X is fluorine or chlorine. In certain embodiments, the substituted alkyl comprises a sulfonyl group (e.g., a sulfone), and may be selected from group consisting of triflate, triflyl, tosyl, and mesyl. Optionally, the substituted alkyl is selected from the group consisting of acyl, alkoxyalkyl, ester, fluoroalkyl, alkylamino, alkylphenyl, and sulfone. Furthermore, when the substituted alkyl is selected from this group, it is often desirable for the substituted alkyl to contain from 1 to 20 carbon atoms, 1 to 15 carbon atoms, 1 to 10 carbon atoms or 1 to 5 carbon atoms.

The term “aryl” as used herein refers to a monovalent radical obtained by removing a hydrogen atom from a carbon atom of an aromatic ring system. Non-limiting examples of aryl groups contemplated by the invention include monovalent radicals derived from anthracene, azulene, benzene, chrysene, coronene, fluoranthene, fluorene, hexacene, hexaphene, indene, naphthalene, and octacene, to name just a few. In certain preferred implementations of the invention, the number of carbon atoms in the aryl group falls in the range of 6 to 30, 6 to 20, or 6 to 12.

The term “substituted aryl,” as used herein, refers to an aryl group, as defined above, in which one or more hydrogen atoms have been replaced independently with different substituents, such as those set forth herein for the definition of “substituted alkyl.” In certain preferred implementations of the invention, the number of carbon atoms in the “substituted aryl” group falls in the range of 6 to 30, 6 to 20, or 6 to 12.

As used herein, the term “heteroaryl” refers to a monovalent radical that is obtained by removing one hydrogen atom from an atom of a heteroaromatic ring system. Non-limiting examples of heteroaryl groups contemplated by the invention include monovalent radicals derived from the removal of hydrogen from benzofuran, benzothiophene, imidazole, indole, oxazole, pyrazine, pyrrolepyridine, quinoline, and thiophene. In certain preferred implementations the number of atoms in the heteroaryl ring system is in the range of 5 to 25 atoms, 5 to 20 atoms, 5 to 15 atoms, or 5 to 10 atoms.

The term “arylalkyl” refers to a linear or branched alkyl group in which one of the hydrogen atoms bonded to a carbon atom is replaced with an aryl group, as defined herein. Non-limiting examples of arylalkyl groups include benzyl, 2-phenylethan-1-yl, naphthylmethyl, 2-naphthylethan-1-yl, naphthobenzyl, 2-naphthophenylethan-1-yl and the like. In certain preferred embodiments, the number of carbon atoms in the arylalkyl group falls in the range of 7 to 40, 7 to 30, 7 to 20, 7 to 15, or 7 to 10. The related term “substituted arylalkyl” refers to an arylalkyl in which one or more hydrogen atoms are independently replaced with substituents, such as the substituents set forth above in connection with the definition of “substituted alkyl.”

“Cycloalkyl” refers to a saturated cyclic monovalent alkyl radical obtained by removing a hydrogen atom from a cycloalkane. Typical cycloalkyl groups include, but are not limited to, monovalent alkyl radicals derived from cyclopropane, cyclobutane, cyclopentane, cyclohexane, and the like. Preferably, the cycloalkyl group is a C₃-C₁₀ cycloalkyl, more preferably a C₃-C₇ cycloalkyl.

In certain preferred embodiments, the minocycline derivative has a structure according to formula (II) or formula (III), as follows:

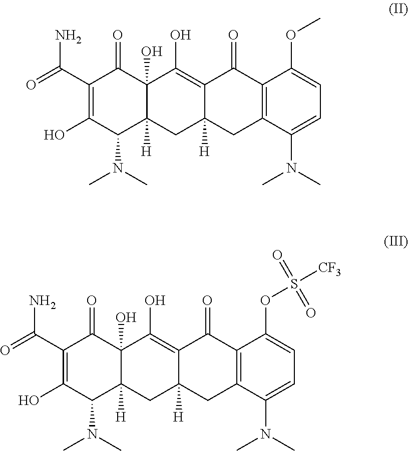 [SMILES: COc1ccc(N(C)C)c2c1C(=O)C1=C(O)[C@]3(O)C(=O)C(C(N)=O)=C(O)[C@@H](N(C)C)[C@@H]3C[C@@H]1C2 ; CN(C)c1ccc(OS(=O)(=O)C(F)(F)F)c2c1C[C@H]1C[C@H]3[C@H](N(C)C)C(O)=C(C(N)=O)C(=O)[C@@]3(O)C(O)=C1C2=O]

Pharmaceutically acceptable salts, tautomers, or stereoisomers of the compounds illustrated by formulas (I)-(III) are also within the scope of the invention. By “pharmaceutically acceptable,” it is meant that a particular component is generally regarded as safe and non-toxic at the levels employed. The compounds of the present invention represented by the above described formulas (I)-(III) may include enantiomers depending on the presence of chiral centers or isomers depending on the presence of multiple bonds (e.g., Z, E). The single isomers, enantiomers or diastereoisomers and mixtures thereof fall within the scope of the present invention.

This invention also includes “pharmaceutically acceptable salts” of the compounds illustrated by formulas (I)-(III). Such salts are synthesized from the parent compound and a basic or acidic moiety by conventional chemical methods. For example, the salts may be prepared by reacting the free acid or base forms of these compounds with a stoichiometric amount of the appropriate base or acid in water or in a non-aqueous media or in a mixture of the two. Nonaqueous media may be without limitation an ether, ethyl acetate, ethanol, isopropanol or acetonitrile. Examples of the acid addition salts include mineral acid addition salts such as, for example, hydrochloride, hydrobromide, hydroiodide, sulfate, nitrate, phosphate, and organic acid addition salts such as, for example, acetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic amino-acid salts. It will be appreciated that non-pharmaceutically acceptable salts also fall within the scope of the invention since those may be useful in the preparation of pharmaceutically acceptable salts. The preparation of appropriate minocycline salts can be carried out by methods known in the art.

The terms “freebase” as used herein refers to compounds according to formula (I) that are not salts. Freebase compounds may be obtained using methods generally known in the art.

The term “solvate” describes a molecular complex comprising the compound and one or more pharmaceutically acceptable solvent molecules (e.g., ethanol). The term “hydrate” refers to a particular form of solvate in which the solvent is water. In a particular embodiment, the solvate is a hydrate. Methods of solvation are generally known within the art. Suitable solvates according to the invention are those that are pharmaceutically acceptable.

The compounds illustrated by formulas (I)-(III), and the pharmaceutically acceptable salts, solvates, and hydrates thereof, may exist in a range of solid states forms from crystalline to polymorphs to amorphous. It is to be understood that the minocycline derivatives of the invention may be in crystalline form either as free compounds, as salts (e.g., with hydrochloric acid), or as solvates (e.g., hydrates) and it is intended that such forms are within the scope of the present invention.

The compounds illustrated by formulas (I)-(III) or their salts or solvates are preferably in pharmaceutically acceptable form. “Pharmaceutically acceptable form” means that the compounds have a pharmaceutically acceptable level of purity excluding normal pharmaceutical additives such as diluents and carriers, and include no material considered toxic at normal dosage levels. Purity levels for the drug substance are preferably above 50%, more preferably above 70%, most preferably above 90%. In a preferred embodiment, the purity is above 95%.

For each of the minocycline derivatives of the invention, the concentration of the minocycline derivative at a particular region within a body (e.g., serum) is dependent on the rates of absorption, conversion (if applicable), and elimination. In certain preferred embodiments, the pharmacokinetic profile of the minocycline derivative is selected to maximize the area under the curve (AUC) and avoid an undesirably high maximum concentration Cₘₐₓ.

In certain preferred embodiments, the minocycline derivatives of the invention are prodrugs that generate minocycline in the body through normal metabolic pathways, but at a rate that reduces the peak plasma concentration as compared to conventional administration of an equivalent amount minocycline. For such minocycline derivatives, the rate of minocycline generation in the body can be estimated using well known in vitro models that are designed to mimic chemical conditions within the body. For example, the rate of minocycline generation from a particular minocycline derivative can be estimated by adding the derivative to simulated gastric fluid (SGF) or simulated intestinal fluid (SIF) prepared in accordance with the United States Pharmacopeia and then analyzing the changes in chemical composition as a function of time. Similarly, the rate of minocycline generation from a particular minocycline derivative in serum may be estimated by adding the minocycline derivative to serum (e.g., fetal calf serum) and monitoring the changes in chemical composition as a function of time. The rate of basal hydrolysis in water also may be used to estimate the rate at which a particular minocycline derivative of the invention is converted to minocycline.

In general, the minocycline derivatives according to the invention may be administered to subjects or patients to treat or prevent infection or inflammatory or non-inflammatory conditions or diseases associated with infection. As used herein, the terms “subject” or “patient” refers to a mammal, including humans, as well as companion animals, such as dogs and cats, and commercial or farm mammals, such as hogs, cattle, horses, goats, sheep, rabbits, etc. “Treating” refers to reversing, alleviating, remediating, and/or inhibiting the progress of a disorder or condition to which such term applies, or to reversing, alleviating, remediating, and/or inhibiting the progress of, or preventing one or more symptoms of such disorder or condition. The term “prevent,” as used herein, includes delaying the onset of or progression of a particular type of condition or disease associated with infection. “Therapeutically effective amount” refers to the quantity of a compound that may be used for treating a subject, which amount may depend on such variables as the subject's biometrics, the route of administration, and the like. It is to be understood that the term “therapeutically effective amount” implicitly refers to an amount that has a beneficial therapeutic effect and is either safe or has an acceptable toxicity profile for the given indication that is to be treated.

Further provided herein are methods of treating conditions or diseases associated with infections in a subject by administering to a subject in need thereof a therapeutically effective amount of one or more of the minocycline derivatives described herein, or a pharmaceutically acceptable salt thereof.

In general, the minocycline derivatives of the invention may be used to treat or prevent any condition that is amenable to treatment with minocycline, particularly infections, (e.g., bacterial infections). For example, the minocycline derivatives of the invention may be used to treat or prevent inflammatory and non-inflammatory conditions or diseases associated with infection, including without limitation, acne, methicillin-resistant Staphylococcus aureus (MRSA), and Lyme disease. In addition, the minocycline derivatives of the invention may be used to treat asthma, rheumatoid arthritis, amoebic dysentery, anthrax, cholera, gonorrhea, Gougerot-Carteaud Syndrome (Confluent and Reticulated Papillomatosis), bubonic plague, perioral dermatitis, periodontal disease, respiratory infections such as pneumonia, HIV (as an adjuvant), secondary infections associated with AIDS, Rocky Mountain spotted fever, rosacea, syphilis, urinary tract infections, rectal infections, and skin infections such as Hidradenitis Suppurativa. In one preferred embodiment, the minocycline derivatives of the invention are be used to treat acne, non-limiting examples of which include acne, acne vulgaris, acne rosacea, acne conglobata, acne fulminans, gram-negative folliculitis, and pyoderma faciale, among others.

The doses of the compounds used in treating the disorders listed herein in accordance with this invention will vary in the usual way with the indication, seriousness of the indication, the biometrics, and health of the individual in need of treatment. The doses for the general patient population may be determined by routine dose-ranging studies, as will be appreciated by a person of ordinary skill in the art. Therapeutically effective doses for individual patients may be determined, by titrating the amount of drug given to the individual to arrive at the desired therapeutic or prophylactic effect, while minimizing side effects.

Useful doses of minocycline derivatives are from about 0.05 to about 5.0 mg/kg/day, from about 0.10 to about 3.0 mg/kg/day, from about 0.15 to about 2.5 mg/kg/day, from about 0.20 to about 2.0 mg/kg/day, from about 0.25 to about 1.0 mg/kg/day, from about 0.5 to about 1.0 mg/kg/day, or from about 0.5 to about 0.75 mg/kg/day. In a preferred embodiment, the dose of minocycline derivatives and/or related compounds is from about 0.10 to about 3.0 mg/kg/day. In another preferred embodiment, the dose of minocycline derivatives and/or related compounds is from about 0.25 to about 2.5 mg/kg/day. In some embodiments, the daily dose of minocycline derivatives and/or related compounds is about 0.05 mg/kg/day, 0.10 mg/kg/day, 0.15 mg/kg/day, 0.20 mg/kg/day, 0.25 mg/kg/day, 0.5 mg/kg/day, 0.75 mg/kg/day, 1.0 mg/kg/day, 1.25 mg/kg/day, 1.5 mg/kg/day, 2.0 mg/kg/day, 2.5 mg/kg/day, 3.0 mg/kg/day, 3.5 mg/kg/day, 4.0 mg/kg/day, 4.5 mg/kg/day, or 5.0 mg/kg/day. Administration schedules may also be altered to achieve a therapeutically effective concentration of compound to treat the disorder or symptoms described herein.

Optionally, minocycline derivatives and/or related compounds may be administered once per day, twice per day, thrice per day, 4 times per day, 5 times per day, 7 times per day or 10 times per day. In a preferred embodiment, minocycline derivatives are administered once per day. Optionally, the dosage is divided equally throughout the day, however in some embodiments to treat certain disorders or symptoms, it may be useful to bias the dosage administration schedule so that most of the daily treatment is administered at the beginning half of the day. In some embodiments, about 50%, 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

In certain embodiments, two or more minocycline derivatives having different pharmacokinetic profiles are administered at the same or different times to tailor the concentration profile of minocycline derivatives in the body as a function of time. Depending on the absorption, conversion, and elimination characteristics of the two or more minocycline derivatives chosen, it may be desirable to administer them together, separately, or serially.

Administration of the compounds of this invention may be by any method used for administering therapeutics, such as for example, oral, topical (such as through the use of a transdermal patch), rectal, or parenteral administration. Suitable parenteral administrations include intravenous, intraarterial, intraperitoneal, intraventricular, intrathecal, intraurethral, intrasternal, intramuscular, intracranial, intrasynovial, and subcutaneous administration via needle injectors, needle-free injectors, microneedle injectors and infusion devices. The minocycline derivatives of the invention may also be administered topically, intradermally, or transdermally to the skin or mucosa. Typical formulations for this purpose include gels, hydrogels, lotions, solutions, creams, liposomes, ointments, dusting powders, dressings, foams, films, skin patches, wafers, implants, sponges, fibers, bandages and microemulsions using carriers and methods known in the art. Preferred administration methods are oral and topical. The most preferred method of administration is oral.

The formulations contemplated by the invention may be in the form of tablets, capsules, powders, granules, lozenges, suppositories, reconstitutable powders, or liquid preparations such as oral or sterile parenteral solutions or suspensions. Thus, in addition to comprising the minocycline derivatives described herein, the therapeutic formulations contemplated by the invention also may comprise a pharmaceutically acceptable carrier. In general, the term “pharmaceutically acceptable carrier” refers to a substance that can be combined with the minocycline derivatives of the invention to form a pharmaceutically acceptable dosage form. Such pharmaceutically acceptable carriers may also improve the stability, ease of administration, and formation of the dosage form used for administration to an individual. Non-limiting examples of such pharmaceutically acceptable carriers include additives, preservatives, excipients, fillers, binders, disintegrants, and buffers. Examples of specific pharmaceutically acceptable carriers contemplated by the invention include, for example, magnesium and/or calcium carbonate, polyvinylpyrrolidone, carboxymethylcellulose, starch (e.g., sodium starch glycolate, microcrystalline cellulose starch, or maize-starch), sugars (e.g., lactose), gums, magnesium and/or calcium stearate, coloring and/or flavoring agents, corn syrup, acacia, gelatin, sorbitol, tragacanth, calcium phosphate, glycine, preservatives, and pharmaceutically acceptable wetting agents such as sodium lauryl sulfate. Optionally, the pharmaceutically acceptable carrier may be saline (e.g., phosphate buffered saline). There exists a wide variety of pharmaceutically acceptable additives for pharmaceutical dosage forms, and selection of appropriate additives is generally a routine matter for those skilled in the art of pharmaceutical formulation.

Optionally, the minocycline derivatives of the invention may be as administered as oral liquid formulations. Such oral liquid formulations may be in the form of, for example, emulsions, syrups, or elixirs. If desired, the oral liquid formulations may be presented as a dry product for reconstitution with water or other liquid pharmaceutically acceptable carrier before use. For example, the oral liquid formulations according to the invention may include methyl sorbitol syrup, carboxymethylcellulose, gelatin, cellulose, hydroxyethylcellulose, aluminum stearate gel, and hydrogenated edible fats, emulsifying agents (e.g., lecithin), sorbitan monooleate, or acacia. If desired, the oral liquid formulations may be prepared using pharmaceutically acceptable carriers that are non-aqueous, including for example, edible oils (e.g., almond oil or fractionated coconut oil), oily esters (e.g., esters of glycerine), propylene glycol, or ethyl alcohol, preservatives, (e.g., methyl or propyl p-hydroxybenzoate or sorbic acid); and conventional flavoring or coloring agents.

Optionally, the minocycline derivatives of the invention may be administered parenterally. For parenteral administration, fluid unit dosage forms may be prepared by combining a minocycline derivative as described herein with a pharmaceutically acceptable liquid carrier. As the skilled artisan will appreciate, the minocycline derivative may be either suspended or dissolved in the pharmaceutically acceptable liquid carrier. For example, in manufacturing parenteral solutions, a minocycline derivative as described herein can be dissolved in a delivery vehicle such as water or saline for injection and filled into a suitable vial or ampoule. Advantageously, additives such as a local anesthetic, preservative and buffering agent can be dissolved in the delivery vehicle. Suitable buffering agents may comprise, for example, phosphate and citrate salts. To enhance the stability, the composition can be lyophilized for storage and reconstituted just prior to administration. Parenteral suspensions are prepared in substantially the same manner, except that the compound is suspended in the vehicle instead of being dissolved. Advantageously, a surfactant or wetting agent is included in the composition to facilitate uniform distribution of the compound.

The invention also expressly contemplates administering the minocycline derivatives described herein using controlled release dosage forms including, but not limited to sustained release dosage forms, extended release dosage forms, delayed release dosage forms, and pulsatile release dosage forms. See e.g., U.S. Pat. No. 8,252,776, which is hereby incorporated by reference in its entirety. Useful solid formulations for oral administration may include immediate release formulations and modified release formulations. Compounds herein, and the pharmaceutically acceptable salts thereof, may also be administered directly into the blood stream, muscle, tissue, or organ system of the subject. Delayed release compositions may be prepared, for example, by employing slow release coatings, micro encapsulation, and/or slowly dissolving polymers. Generally, carriers for controlled release formulations are generally known in the art. For instance, suitable polymers for use as pharmaceutically acceptable carriers in the controlled release formulations of the present invention include, but are not limited to uncrosslinked, linear polymers including cellulosic polymers, preferably hydroxyethyl cellulose, sodium carboxymethyl cellulose, hydroxypropylmethyl cellulose and hydroxypropyl cellulose, microcrystalline cellulose, methyl cellulose, and ethyl cellulose, and combinations thereof; covalently crosslinked insoluble polymers such as high molecular weight crosslinked homopolymers and copolymers of (meth)acrylic acid including carbopol resins, or mixtures of these uncrosslinked and covalently crosslinked polymers. Additionally suitable polymers include acrylic acid, methacrylic acid, methyl acrylate, ammonio methylacrylate, ethyl acrylate, methyl methacrylate and/or ethyl methacrylate, vinyl polymers and copolymers such as polyvinyl pyrrolidone, polyvinyl acetate, polyvinylacetate phthalate, vinylacetate crotonic acid copolymer, and ethylene-vinyl acetate copolymers, to name a few. If desired, two or more of the above polymers may be used in the dosage forms of the invention.

Optionally, the minocycline derivatives of the invention may be provided as part of a kit. In one embodiment, a kit may comprise at least one minocycline derivative, and at least one additional therapeutic compound. Optionally, the kits as described herein may also include instructions for administration of the minocycline derivatives. For example, an exemplary anti-acne kit according to the invention may comprise at least one minocycline derivative, at least one other anti-acne drug, and instructions for administering the compounds.

In another aspect of the invention, methods are provided for preparing a compound according to formula (I), wherein R₁ is defined in formula (I) or a pharmaceutically acceptable salt, tautomer, or stereoisomer thereof. The methods provided herein comprise reacting a minocycline derivative with a proton extraction agent and a derivatizing agent, and isolating the compound, which is a derivative of minocycline, as shown below in Scheme 1.

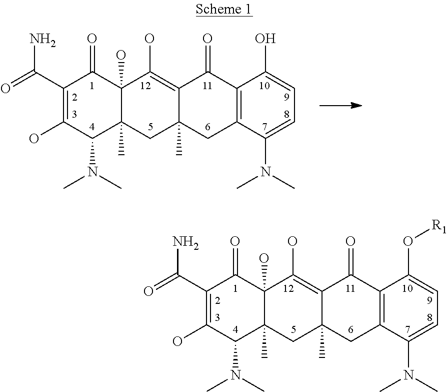

One of ordinary skill in the art will appreciate that the proton extraction agent contemplated by the invention is a compound that removes a hydrogen atom from a particular hydroxyl group on a substituted minocycline molecule, particularly at the C₁₀ hydroxyl. Non-limiting examples of suitable proton extraction agents include, for example, sterically hindered non-nucleophilic bases. Non-limiting examples of such bases include 2,6-di-tert-butylpyridine, potassium t-butoxide, sodium t-butoxide, N,N-diisopropylethylamine, and 1,8-diazabicycloundec-7-ene.

Furthermore, compounds according to formula (I) may be prepared by reacting a deprotonated minocycline intermediate with a derivatizing agent. In this context, a “derivatizing agent” is any reagent that will react with a deprotonated minocycline (particularly those that are deprotonated at the C₁₀ position) to form a minocycline derivative having an R₁ group as described herein. Non-limiting examples of suitable derivatizing agents include methyl-para-toluene sulfonate and N-phenyl-bis(trifluoromethanesulfonimide).

Typically, the chemical reactions described in this invention may be carried out using substantially stoichiometric amounts of reactants, though certain reactions may benefit from using an excess of one or more of the reactants. Additionally, many of the reactions disclosed throughout the specification may be carried out at about room temperature and ambient pressure, but depending on reaction kinetics, yields, and the like, some reactions may be run at elevated pressures or employ higher (e.g., reflux conditions) or lower (e.g., −80° C. to 0° C.) temperatures. Any reference in the disclosure to a stoichiometric range, a temperature range, a pH range, etc., whether or not expressly using the word “range,” also includes the indicated endpoints.

Scheme (1) as described herein may also employ one or more compatible solvents, which may influence the reaction rate and yield of the minocycline derivatives of the invention. Preferably, the chosen solvent will be able to dissolve minocycline as well as all of the other reagents required to synthesize the minocycline derivative in question, so that the reaction can occur in one reaction vessel without the need for additional isolation and purification steps. The solvent may be a polar protic solvent (including water), a polar aprotic solvent, a non-polar solvent, or combinations thereof. Optionally, the solvent may be an aprotic solvent capable of dissolving both polar and non-polar compounds, a non-limiting example of which is tetrahydrofuran. In some embodiments, one or more solvents may be used either in sequence or together in the synthesis of the minocycline derivatives of the invention.

In one aspect, the invention provides a method for treating acne. Acne, a familiar skin disease which appears during adolescence and extends into adulthood, afflicts about 85% of people during their lives. Acne is a disorder of the sebum producing hair follicles, or pores, and it affects various areas of the body including face, chest, back, neck and scalp. Sebum is an oily substance, which keeps the skin flexible, supple, and moist, and drains to the surface of the skin. Usually, skin regenerates, by shedding old cells. However, when cells are irregularly shed, they cluster together with the sebum, and plug skin's pores. This plugging leads to a rapid growth of bacteria (e.g., Propionibacterium acnes), which together with the accumulation of sebum, enlarges the hair follicles and results in a type of acne called comedones (e.g., whiteheads and blackheads). In general, the factors causing acne are increased sebum production, bacteria, hormones, and changes inside of the hair follicle. The transformation of acne to a more severe inflammatory type, is accompanied by the presence of red lesions called papules, pustules and nodules. Among the most common types of acne are acne vulgaris, acne rosacea, acne conglobata, acne fulminans, gram-negative folliculitis, and pyoderma faciale.

The method for treating acne comprises administering one or more minocycline derivatives according to the invention to a patient in need of treatment. Optionally, the minocycline derivatives may be formulated for either oral administration or topical administration. The minocycline derivatives may be administered in conjunction with other anti-acne agents, non-limiting examples of which include benzoyl peroxide, salicylic acid, sulfur, resorcinol, clindamycin, erythromycin (and erythromycin salts such as estolate, ethylsuccinate, gluceptate, lactobionate, stearate), tetracycline, doxycycline, cefadroxil, cephadrine, cefazolin, cephalexin, cephalothin, cephapirin, cephacelor, penicillin V, penicillin salts, and complexes.

If desired, the minocycline derivatives may be part of a kit. For instance, when the kit is an anti-acne kit, the additional therapeutic compound may be an anti-acne therapeutic compound. Alternatively, the kit may contain a minocycline derivative according to the invention, along with a cosmetic agent that helps to minimize the appearance of the acne. The kit may also include compositions or patches that act as sealing layers, skin cleansers, skin penetration enhancers, and nutritional supplements. Various anti-acne kits can be customized for spot treatment versus more diffuse treatment, for different skin types, and for night versus day treatment. The anti-acne kits according to the invention can additionally be included as a part of a larger kit or skin care regimen that includes tailored cleansers, toners, balancers, moisturizers and/or various cosmetic or therapeutic topical agents.

### EXAMPLES

The following examples describe specific aspects of the invention to illustrate the invention but should not be construed as limiting the invention, as the examples merely provide specific methodology useful in the understanding and practice of the invention and its various aspects.

### Example 1

### Preparation of Minocycline Freebase

Minocycline hydrochloride was added to a separatory funnel containing H₂O and dichloromethane. To this was added saturated sodium bicarbonate until the pH was 8-9 by pH paper and the funnel was vigorously shaken. The organic layer was then separated and the aqueous layer was washed 4 times with dichloromethane. The organic layer was dried with magnesium sulfate, filtered, and concentrated to give the freebase of minocycline as a bright orange solid.

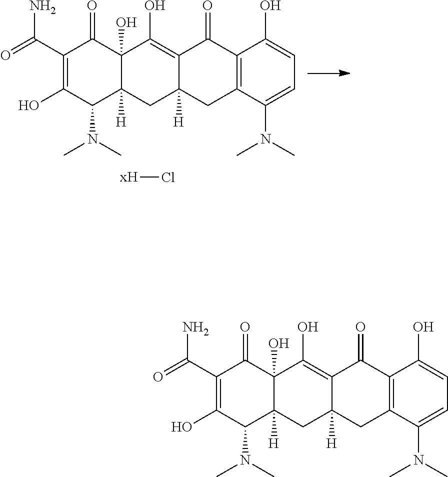

### Example 2

### Preparation of 10-Methoxy-Minocycline

To a 250 ml Schlenk flask was added (1 equiv., 2.0 g, 4.4 mmol) of the freebase of minocycline, as described in Example 1. The flask was then vacuum purged 3 times with argon and to this was added 60 ml of anhydrous tetrahydrofuran. The solution was then cooled to −78° C. with an acetone/CO₂ bath and potassium tert-butoxide (3.66 equiv., 1.85 g, 16.5 mmol) was added in small portions over 10 minutes while maintaining positive argon pressure. The cooling bath was removed and the reaction was allowed to warm to ambient temperature for 45 min (the reaction was an opaque bright yellow). Once at ambient temperature, methyl-para-toluene sulfonate (2.75 equiv, 2.24 g, 12 mmol) was added via syringe over 5 min. The reaction was then stirred overnight, ˜12-16 hours. Once determined to be complete by liquid chromatography-mass spectrometry (LCMS) the reaction was quenched with 300 ml H₂O, 300 ml dichloromethane, and carefully brought to pH 7.5 with 1N HCl. The layers were separated and the aqueous layer was washed three times with 250 ml dichloromethane. The organic layer was dried with magnesium sulfate, filtered, and concentrated to give the crude material as a dark viscous oil. To this oil was added 100 ml hexanes and then heated to a boil. The hexanes were decanted off and the oil dried under vacuum.

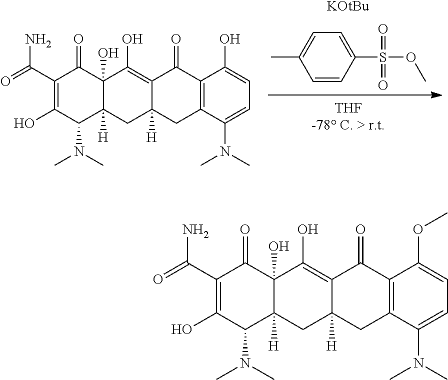

The oil was purified by reverse phase high-performance liquid chromatography (HPLC) utilizing a 5-70% acetonitrile:H₂O gradient to give the TFA salt of 10-methoxy-minocycline as a greenish yellow solid (450 mg, 17.4% yield). ¹H-NMR (DMSO-d₆) δ 9.90 (br s, 1H), 9.50 (br s, 1H), 9.10 (br s, 1H), 7.50 (d, 1H), 7.40, (br s, 1H), 7.05 (m, 1H), 7.1 (d, 1H), 4.20 (s, 1H), 3.80 (s, 3H), 3.70 (s, 1H), 3.40 (s, 1H), 3.35 (s, 2H), 3.3-3.1 (m, 3H), 2.9-2.5 (m, 8H), 2.1 (m, 1H), 1.5 (m, 1H). LCMS retention time 0.600, M+H=472.2.

### Example 3

### Preparation of 10-Trifluoromethylsulfonate-Minocycline

To a 250 ml Schlenk flask was added (1 equiv., 2.0 g, 4.4 mmol) of the freebase of minocycline, as described in Example 1. The flask was then vacuum purged 3 times with argon and to this was added 45 ml of anhydrous tetrahydrofuran. The reaction was cooled to 0° C. with an ice bath and solid potassium tert-butoxide (3.5 equiv., 1.73 g, 15.4 mmol) was added in small portions over 5 minutes while maintaining positive Argon pressure. The reaction was stirred at 0° C. for 35 minutes and then N-phenyl-bis(trifluoromethanesulfonimide) (2.5 equiv., 3.93 g, 11 mmol) was added all at once along with 4-(dimethylamino)pyridine (cat., 22 mg). The reaction was then stirred overnight, ˜12-16 hours. Once determined to be complete by LCMS the reaction was quenched with 300 ml H₂O, 300 ml dichloromethane, and carefully brought to pH 7.5 with 1N HCl. The layers were separated and the aqueous layer was washed 3 times with 250 ml dichloromethane. The organic layer was dried with magnesium sulfate, filtered, and concentrated to give the crude material as a dark brown solid.

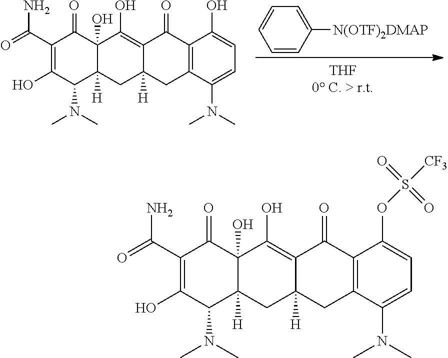

The solid was then purified by reverse phase HPLC utilizing a 5-95% acetonitrile: H₂O gradient to give the TFA salt of 10-trifluoromethylsulfonate-Minocycline as a yellow solid (1.6 g, 51.8% yield). ¹H-NMR (DMSO-d₆) δ 9.90 (br s, 1H), 9.55 (br s, 1H), 9.05 (br s, 1H), 7.60 (br s, 1H), 7.45 (d, 1H), 7.35 (d, 1H), 4.25 (s, 1H), 3.2-2.7 (m, 10H), 2.65 (s, 6H), 2.45 (m, 1H), 2.20 (m, 1H), 1.55 (m, 1H). LCMS retention time 1.120, M+H=590.1.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A compound having a structure according to formula (I) 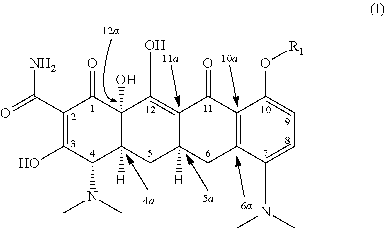
wherein r₁ is selected from the group consisting of alkyl, substituted alkyl, and heteroaryl,
or a pharmaceutically acceptable salt, tautomer, or stereoisomer thereof.

2. The compound according to claim 1, wherein R¹ is alkyl.

3. The compound according to claim 2, wherein the alkyl comprises 1 to 20 carbon atoms.

4. The compound according to claim 3, wherein the alkyl comprises 1 to 10 carbon atoms.

5. The compound according to claim 4, wherein the alkyl comprises 1 to 5 carbon atoms.

6. The compound according to claim 5, wherein the alkyl comprises 1 to 3 carbon atoms.

7. The compound according to claim 2, wherein the alkyl is selected from the group consisting of methyl, ethyl, propan-1-yl, propan-2-yl, cyclopropan-1-yl; butan-1-yl, butan-2-yl, 2-methyl-propan-1-yl, 2-methyl-propan-2-yl, and cyclobutan-1-yl.

8. The compound according to claim 7, wherein the alkyl is methyl or ethyl.

9. The compound according to claim 8, wherein the alkyl is methyl.

10. The compound according to claim 1, wherein R₁ is a substituted alkyl.

11. The compound according to claim 10, wherein the substituted alkyl contains 1 to 20 carbon atoms.

12. The compound according to claim 11, wherein the substituted alkyl contains 1 to 15 carbon atoms.

13. The compound according to claim 12, wherein the substituted alkyl contains 1 to 10 carbon atoms.

14. The compound according to claim 13, wherein the substituted alkyl contains 1 to 5 carbon atoms.

15. The compound according to claim 10, wherein the substituted alkyl is selected from the group consisting of —X, —CX₃, —OR², —C(O)R², —C(S)R², —C(O)OR², —C(O)NR²R³, —SR², —S—, ═S, —O—, ═O, —CN, —OCN, —SCN, —NO, NO₂, S(O)₂O, —NR²R³, ═NR², —S(O)₂OH, —S(O)₂R², —R²S(O)₂R³, —OS(O)₂R², and —S(O)₂CX₃,
wherein each X is independently a halogen, and each R² and R³ are independently hydrogen, alkyl, aryl, substituted aryl, arylalkyl, substituted arylalkyl, and cycloalkyl.

16. The compound according to claim 10, wherein the substituted alkyl is selected from the group consisting of acyl, alkoxyalkyl, ester, fluoroalkyl, alkylamino, alkylphenyl, and sulfone.

17. The compound according to claim 10, wherein the substituted alkyl comprises a sulfonyl group.

18. The compound according to claim 17, wherein the substituted alkyl is selected from the group consisting of triflate, triflyl, tosyl, and mesyl.

19. The compound according to claim 1, wherein R₁ is a heteroaryl.

20. The compound according to claim 19, wherein the number of atoms in the heteroaryl ring system is in the range of 5 to 25 atoms.

21. The compound according to claim 20, wherein the number of atoms in the heteroaryl ring system is in the range of 5 to 20 atoms.

22. The compound according to claim 21, wherein the number of atoms in the heteroaryl ring system is in the range of 5 to 15 atoms.

23. The compound according to claim 22, wherein the number of atoms in the heteroaryl ring system is in the range of 5 to 10 atoms.

24. A pharmaceutical formulation comprising a compound with a structure according to formula (I) 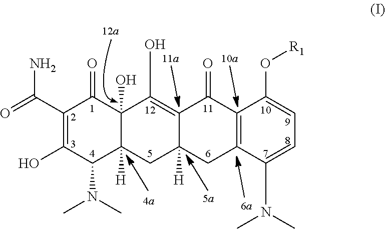
wherein R₁ is selected from the group consisting of alkyl, substituted alkyl, and heteroaryl,
or a pharmaceutically acceptable salt, tautomer, or stereoisomer thereof,
and a pharmaceutically acceptable diluent or carrier.

25. The pharmaceutical formulation according to claim 24, wherein the compound is selected from the group consisting of

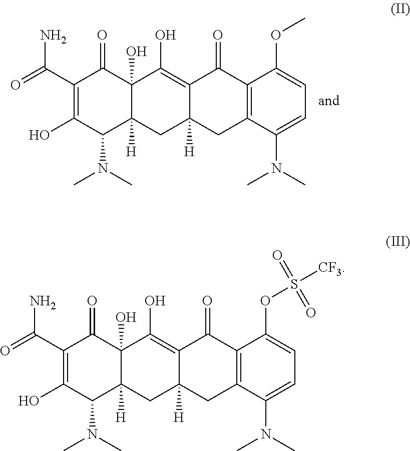 [SMILES: COc1ccc(N(C)C)c2c1C(=O)C1=C(O)[C@]3(O)C(=O)C(C(N)=O)=C(O)[C@@H](N(C)C)[C@@H]3C[C@@H]1C2 ; CN(C)c1ccc(OS(=O)(=O)C(F)(F)F)c2c1C[C@H]1C[C@H]3[C@H](N(C)C)C(O)=C(C(N)=O)C(=O)[C@@]3(O)C(O)=C1C2=O]

26. The pharmaceutical formulation according to claim 24, wherein the pharmaceutical formulation is administered orally.

27. The pharmaceutical formulation according to claim 24, wherein the pharmaceutical formulation is administered topically.

28. The pharmaceutical formulation according to claim 24, wherein the pharmaceutical formulation is administered by injection or intravenously.

29. A method of treating or preventing an infection, the method comprising
administering to a patient in need thereof a therapeutically effective amount of a pharmaceutical formulation comprising a compound with a structure according to formula (I) 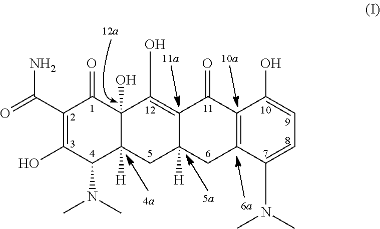
wherein R₁ is selected from the group consisting of alkyl, substituted alkyl, and heteroaryl, or a pharmaceutically acceptable salt, tautomer, or stereoisomer thereof.

30. The method according to claim 29, wherein the pharmaceutical formulation is administered orally.

31. The method according to claim 29, wherein the pharmaceutical formulation is administered topically.

32. The method according to claim 29, wherein the pharmaceutical formulation is administered by injection or intravenously.

33. The method according to claim 29, wherein the infection is selected from the group consisting of acne, methicillin-resistant Staphylococcus aureus (MRSA) infection, Lyme disease, amoebic dysentery, anthrax, cholera, gonorrhea, Gougerot-Carteaud Syndrome (Confluent and Reticulated Papillomatosis), bubonic plague, perioral dermatitis, periodontal disease, respiratory infections, a secondary bacterial infection associated with AIDS, Rocky Mountain spotted fever, rosacea, syphilis, urinary tract infection, rectal infection, and skin infection.

34. The method according to claim 29, wherein the infection is an acne condition selected from the group consisting of acne vulgaris, acne rosacea, acne conglobata, acne fulminans, gram-negative folliculitis, and pyoderma faciale.

35. A method of treating an inflammatory condition, the method comprising
administering to a patient in need thereof a therapeutically effective amount of a pharmaceutical composition comprising a compound with a structure according to formula (I) 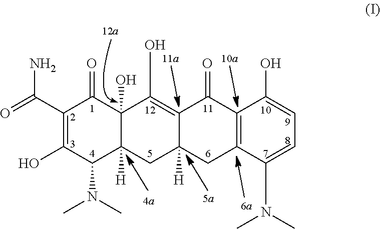
wherein R₁ is selected from the group consisting of alkyl, substituted alkyl, and heteroaryl, or a pharmaceutically acceptable salt, tautomer, or stereoisomer thereof,
wherein the inflammatory condition is selected from the group consisting of asthma and rheumatoid arthritis.

36. A method of treating or preventing an infection, the method comprising
administering to a patient in need thereof a therapeutically effective amount of a pharmaceutical composition comprising two or more different compounds, each having a structure in accordance with formula (I): 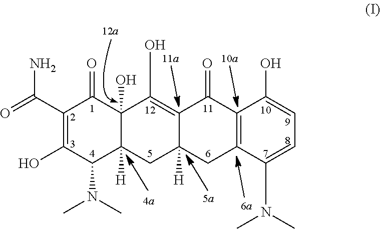
wherein R₁ is selected from the group consisting of alkyl, substituted alkyl, and heteroaryl, or pharmaceutically acceptable salts, tautomers, or stereoisomers of the two different compounds,
wherein the in vivo pharmacokinetic spectra of the two or more different compounds are not equal.

37. A method of synthesizing a compound with a structure according to formula (I) 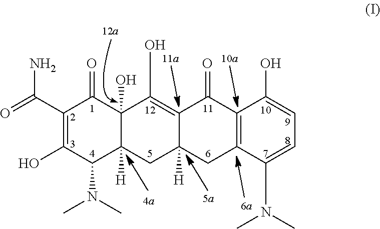 [SMILES: CN(C)c1ccc(O)c2c1C[C@H]1C[C@H]3[C@H](N(C)C)C(O)=C(C(N)=O)C(=O)[C@@]3(O)C(O)=C1C2=O]
the method comprising the steps of:
reacting minocycline with a deprotonating agent to form a minocycline intermediate; and
reacting the minocycline intermediate with a derivatizing agent.

38. The method according to claim 37, wherein the deprotonating agent is selected from the group consisting of 2,6-di-tert-butylpyridine, potassium t-butoxide, sodium t-butoxide, N,N-diisopropylethylamine, and 1,8-diazabicycloundec-7-ene.

39. The method according to claim 37, wherein the derivatizing agent is selected from the group consisting of methyl-para-toluene sulfonate and N-phenyl-bis(trifluoromethanesulfonimide).

40. A kit comprising
a compound having a structure according to formula (I) 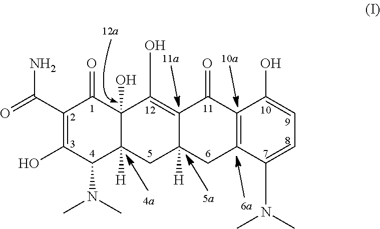
wherein R₁ is selected from the group consisting of alkyl, substituted alkyl, and heteroaryl,
or a pharmaceutically acceptable salt, tautomer, or stereoisomer thereof; and
instructions for administering the compound.
